(19) United States
(12) Patent Application Publication
(10) Pub. No.: US 2004/0096457 A1
(43) Pub. Date: May 20, 2004

(54) Treatment and prevention of ebv infection and ebv-associated disorders

(76) Inventors: Brigitte T Huber, Cambridge, MA (US); David A Thorley-Lawson, Cambridge, MA (US); Natalie Sutkowski, Gloucester, MA (US)

Correspondence Address: NIXON PEABODY LLP, ATTENTION: DAVID RESNICK, 101 FEDERAL STREET, BOSTON, MA, 02110, US

(21) Appl. No.: 10/432,114

(22) PCT Filed: Dec. 11, 2001
(86) PCT No.: PCT/US01/47885

(51) Int. Cl. A61K 39/12

(52) U.S. Cl. 424/186.1

(57) ABSTRACT

The present invention provides substances suitable for use as vaccines for the prevention of EBV infection and EBV-associated disorders and methods for administering them. The vaccines are directed against HERV-K18 emv (SEQ ID:1) and most preferably comprise antigens obtained from HERV-K18 emv. Preferred antigens include SEQ ID:2, SEQ ID:3 and SEQ ID:4. Most preferably, the SAg T cell stimulatory activity of the HERV-K18 emv is diminished or eliminated. In another embodiment, the vaccine contains a nucleic acid encoding HERV-K18 emv or an immunogenic fragment thereof. The present invention also provides methods for treating EBV infection and EBV-associated disorders.

### CROSS-REFERENCE TO RELATED APPLICATION

This Application is based on Provisional Application 60/254,673, filed 11 Dec. 2000, the content of which is relied upon and incorporated herein by reference in its entirety, and benefit priority under 35 U.S.C. §119(e) is hereby claimed.

### GOVERNMENT FUNDING

This invention was made with government support under AI14910 awarded by

the National Institutes of Health. The government has certain rights in the invention.

### FIELD OF THE INVENTION

The present invention relates to a method of treatment and prevention of Epstein-Barr Virus (EBV) infection and EBV-associated disorders.

### BACKGROUND

EBV is a ubiquitous human herpesvirus which infects the majority of the population and is associated with disease and neoplasia. A double-stranded DNA virus of 172 kb, EBV can infect lymphocytes and epithelial cells. Infection of B lymphocytes with EBV results in their activation and proliferation. More than 90% of adults are latently infected with EBV. In most individuals primary EBV infection occurs during childhood and does not result in clinical manifestations. If primary infection is delayed until adolescence, infectious mononucleosis (1M), a self-limiting proliferation of EBV-infected B cells, can result.

Subsequent to primary infection, EBV-infected cells persist within the host for life. Low levels of infectious virus are shed into the saliva in most asymptomatic seropositive individuals. EBV-infected B cells are kept from proliferating out of control in vivo by a properly functioning immune system. In individuals who are immunosuppressed, however, EBV-infected cells can give rise to lymphoproliferative disorders leading to disease or oncogenesis.

EBV infection is known to be associated with a number of pathological conditions, including X-linked lymphoproliferative syndrome (XLP), malignancies such as nasopharyngeal carcinoma (NPC), endemic Burkitt's Lymphoma (BL) and Hodgkin's Disease (HD) (reviewed in Rickinson et al., Virology, Fields et al., eds., 3d ed. 1996, pp. 2397-2446, Lippincott-Raven, Philadelphia, Pa.). Additionally, 50% of breast cancer have recently been shown to be EBV positive (Gonnet M., Guinebrettiere J-M., Kremmer E., Grunewald V., Benhamou E,. Contesso G., Joab I. Detection of Epstein-Barr Virus in invasive Breast Cancers. J. Nat. Cancer Inst. 91: 1376-81, 1999) and autoimmume diseases such as lupus (Harley J. B., James J. A. Epstein-Barr virus infection may be an environmental risk factor for systemic lupus erythematosus in children and teenagers. Arthritis Rheum. 1999 August; 42(8):1782-3), rheumatoid arthritis (Takeda T., Mizugaki Y., Matsubara L., Imai S., Koike T., Takada K. Lytic Epstein-Barr virus infection in the synovial tissue of patients with rheumatoid arthritis. Arthritis Rheum. 2000 June;43(6):1218-25) and Sjogren's syndrome (Saito I. B., Servenius T., Compton T., Fox R. I. Detection of Epstein-Barr virus DNA by polymerase chain reaction in blood and tissue biopsies from patients with Sjogren's syndrome. J. Exp. Med. 169: 2191-98, 1989), have been also linked to EBV. Further, immunosuppressed individuals, such as organ transplant recipients being treated with immunosuppressive drugs, can develop EBV-positive B cell lymphomas. Individuals infected with human immunodeficiency virus (HIV) can also develop EBV-positive B cell lymphomas, which are called AIDS-related lymphomas (ARLs). Oral hairy leukoplakia (OHL), which manifests itself as EBV-infected epithelial lesions on the tongue, has also been observed in AIDS patients.

High EBV titers, as well as high levels of T cells (e.g., Vβ13 T cells) have been reported in individuals suffering from EBV-associated autoimmune diseases, such as rheumatoid arthritis or Sjogren's syndrome (Saito et al., J. Exp. Med. 169: 2191-2198 (1989); Saito et al., J. Exp. Med. 169: 2191-2198 (1989); Sumida et al., J. Clin. Invest. 89: 681-685 (1992); Yonaha et al., Arthritis Rheum. 35: 1362-1367 (1992); and Sumida et al., Br. J. Rheumatol. 33: 420-424 (1994)).

Pathogenic microbes are known to produce certain proteins, called Superantigens (SAgs), which elicit potent, antigen-independent T cell response that is believed to enhance the microbes' pathogenicity. There are two groups of microorganisms, bacterial and viral, that are known to have SAgs. While a large number of bacterial SAgs have been well characterized structurally and functionally, only three families of viruses have been associated with SAg activity to date: retroviruses, rhabdovirus and herpesviruses. Huber, B. T., Hsu, P. N. & Sutkowski, N. Virus-encoded superantigens. Microbiol Rev, 60, 473-482 (1996).

We have reported previously that EBV-infected-B cells express a SAg and proposed that SAg-mediated T cell activation contributes to the lymphocytosis seen during infectious mononucleosis (IM), a disease associated with acute EBV infection. (Sutkowski, N. et al. An Epstein-Barr virus-associated superantigen. J Exp Med 184, 971-980 (1996); Reinherz, E. L., O'Brien, C., Rosenthal, P. & Schlossman, S. F. The cellular basis for viral-induced immunodeficiency: analysis by monoclonal antibodies. J Immunol 125, 1269-1274 (1980); Henle, G., Henle, W. & Diehl, V. Relation of Burkitt's tumor-associated herpes-type virus to infectious mononucleosis. Proc Natl Acad Sci USA 59, 94-101 (1968)). As mentioned earlier, SAg driven T cell activation facilitates progression of EBV infection towards lifelong viral persistence in the resting memory B cell compartment and/or plays a role in viral reactivation. Miyashita. E. M., Yang, B., Lam, K. M., Crawford, D. H. & Thorley-Lawson, D. A. A novel form of Epstein-Barr virus latency in normal B cells in vivo. Cell 80, 593-601 (1995); Hasuike, S. et al. Isolation and localization of an IDDMK1,2-22-related human endogenous retroviral gene, and identification of a CA repeat marker at its locus. J Hum Genet 44. 343-347 (1999).

SAgs are microbial pathogen-derived proteins that evoke a strong T cell response from the host. They do this by associating with MHC class II molecules and binding to T cells that express particular T cell receptor (β-chain variable TCRBV) genes. This distinguishes them from specific antigens that bind to the groove formed by the α and β, chains of the TCR and, thus, activate a small population of T cells only.

It is believed that the T cell stimulation elicited by SAgs does not limit the pathogen, as would a normal T cell response. Paradoxically, the response seems to be beneficial, helping the pathogen to complete its life cycle. We found previously a T cell receptor β chain variable (TCRBV13) gene specific SAg activity associated with the ubiquitous herpesvirus Epstein-Barr virus (EBV). Sutkowski, N. et al. An Epstein-Barr virus-associated superantigen. J Exp Med 184, 971-980 (1996). We now discovered that this SAg is encoded by the env gene of an endogenous retrovirus, HERV-K18, which is transactivated by EBV. This is the first report of an infectious agent borrowing a host encoded SAg. It appears that EBV uses this T cell stimulatory activity to facilitate the establishment of persistent infection in B cells. Deregulation of SAg mediated T cell activation is crucial in the pathogenesis of infectious mononucleosis, the EBV-associated malignancies and EBV-associated autoimmune disorders, many of which are characterized by large T cell infiltrates.

Different approaches have been used to attempt to reduce pathology associated with EBV infection. For example, pyrophosphate analogs, thymidine kinase analogs, ribonucleoside reductase inhibitors, and nucleoside analogs, such as acyclovir, have been used to control diseases associated with EBV infection. None of these agents are effective against latent EBV, nor ideal for inhibiting EBV replication and associated pathology. In addition, use of these agents can result in inhibition of normal cellular processes, which in turn results in undesirable side effects. Antisense oligodeoxynucleotides have also been designed that are specific for various EBV genes, which are associated with the EBV lytic and latent cycles. U.S. Pat. No. 5,242,906 and U.S. Pat. No. 5,837,854; Roth et al., Blood 84: 582-587 (1994); WO 93/11267.

Therefore, there remains a great need for effective prevention and treatment of EBV infection and EBV-associated disorders.

### SUMMARY OF THE INVENTION

We have discovered that EBV infection leads to induction of an endogenous retrovirus that expresses T cell superantigen (SAg) activity which, in turn, rapidly progresses into polyclonal T cell activation with widespread implications for EBV pathogenesis. For example, massive T cell infiltrates are characteristic of the EBV-associated tumors such as Hodgkin's lymphoma and naso-pharyngeal carcinoma; and activated T helper cells play a role in the development of transplant associated lymphomas. Furthermore, massive lymphocytosis is a characteristic of acute IM. Therefore, without wishing to be bound by theory, we believe that EBV induced SAg activity plays a role in a long list of diseases associated with these processes and that prevention or inhibition of such activity would be useful in treating and/or preventing EBV infection and EBV-associated disorders.

One embodiment of the invention provides a method of vaccination for prevention and treatment of EBV infection and EBV-associated disorders. Such method includes a vaccine for treating and/or preventing EBV infection and EBV-associated disorders comprised of HERV-K18 env (SEQ ID:1) or an immunogenic fragment thereof, or a nucleic acid encoding the HERV-K18 env, or a fragment thereof and a pharmaceutically acceptable carrier.

Another embodiment of the invention provides a method for preventing EBV infection and EBV-associated disorders in an individual at risk for such infection or disorder comprising administering to such individual a vaccine comprising a peptide having the amino acid sequence of SEQ ID: 1, SEQ ID:2 (cpkeipkgskntevl), SEQ ID:3, and SEQ ID:4.

In a preferred embodiment, the HERV-K18 env or immunogenic fragment thereof has a diminished or eliminated SAg T cell stimulatory activity. As used herein the term “diminished” means that the SAg T cell stimulatory activity is reduced by at least 50% compare to the normal, more preferably by at least 75%, and even more preferably by 95%. Sag T cell stimulatory activity can be measured as described more fully in the Examples infra. SAg T cell stimulatory activity can be diminished, and preferably eliminated, using standard techniques including amino acid substitutions, additions and deletions. SAg T cell stimulatory activity can be tested against VB13+T cells.

Yet another embodiment of the invention provides a method for treating an individual having an EBV-associated disorder, such as IM and EBV-induced lymphomas, and includes administering to such individual a treatment effective amount of an antibody or a fragment thereof against HERV-K18 env. The antibody fragments include, for example, Fab, Fab′, F(ab′)2 or Fv fragments. The antibody may be a single chain antibody, a humanized antibody or a chimeric antibody. In adolescents, for example, the recovery period for IM is protracted, often lasting for a period of months. However, early identification of the disease, followed by the administration of a pharmaceutical composition comprising the antibody which would block activation of HERV-K18 env SAg, would reduce the duration and severity of the disease. Early identification of IM is accomplished by administering, for example, a monospot or an EBV specific serological test to individual presenting common symptoms of the disease (e.g., swollen glands, sore throat, etc.).

Yet another embodiment of the invention provides a method of passive immunotherapy to infection by EBV in an individual susceptible to infection by EBV. This method involves administering to said individual a HERV-K18 env antibody composition.

In a further embodiment, we provide a method for treating and/or preventing oncogenic transformation in immunocompromised (immunosuppressed) individual. The method includes identifying immunocompromised individuals exhibiting clinical symptoms associated with early stage oncogenic transformation, and administering to such individuals, a therapeutically effective amount of a vaccine comprising a peptide having the amino acid sequence of SEQ ID:1, SEQ ID:2, SEQ ID:3, and SEQ ID:4 or an antibody or a fragment thereof against HERV-K18 emv. The antibody or a fragment thereof may be administered before the commencement of immunosuppressive therapy. Preferably, the antibody administration continues throughout the immunosuppressive therapy. The oncogenic transformation can result in lymphomas including Hodgkin's lymphoma, Post-transplant-lymphoproliferative disorders Lympho-proliferative Disorders, EBV-positive breast cancer, Burkitt's lymphoma, and Naso-Pharyngeal-Carcinoma.

Immunocompromised (immunosuppressed) individuals are characterized by a general depletion of T cell function. Reactivation of EBV in such individuals has been linked to oncogenesis. Therefore, by preventing or interfering with HERV-K18 env SAg activity EBV-induced oncogenesis can be eliminated, or substantially reduced.

Immunosuppression can arise in a variety of ways. For example, many pathogens suppress immune responses in general. HIV infection represents an extreme case of pathogen-induced immune suppression. The ultimate cause of death in AIDS is usually infection with an opportunistic pathogen (a pathogen which is present in the environment but does not usually cause disease because it is controlled by the normal immune response). Therefore, in the case of an individual suffering from pathogen-induced immune suppression, the administration of an antibody or a fragment thereof against HERV-K18 env, would be indicated for the duration of the pathogen-induced immunosuppression.

Medically-induced immunosuppression (iatrogenic immunosuppression) is required, for example, in connection with organ and bone marrow transplant. Cyclosporin A is widely used in clinical transplantation because it is both effective and relatively non-toxic. An unrelated compound with similar activity is FK506. These compounds prevent the synthesis of IL-2 by blocking a late stage of the signaling pathway initiated by the T cell receptor.

Individuals receiving organ transplants are acutely immunosuppressed (i.e., immunoincompetent) for some period of time (e.g., one to several months) following solid organ transplant. Following this period of acute immunosuppression, a degree of immunocompetence is allowed to establish, although a basal level of immunosuppression is generally maintained for the lifetime of the individual. To prevent oncogenic transformation in such individuals, the administration of a pharmaceutical composition comprising an antibody or a fragment thereof against HERV-K18 env is provided in the present invention. Preferably, the period of administration is the period of acute immunosuppression.

Bone marrow transplant recipients also require a period of immunosuppression following transplant. The period of immunosuppression is required to permit repopulation of the transplanted cells. During this period of immunosuppression, the administration of a pharmaceutical composition comprising an antibody or a fragment thereof against HERV-K18 env, would prevent EBV induced lymphomas.

In yet another embodiment, a method of treating an EBV-associated autoimmune disorder is also provided. The method involves identifying an EBV-positive individual acutely afflicted with an autoimmune disorder and administering to such individual, an effective amount of an antibody or a fragment thereof against HERV-K18 env.

Finally, there is provided an article of manufacture comprising packaging material and a pharmaceutical agent contained within said packaging material, wherein said packaging material comprises a label which indicates said pharmaceutical may be administered, for a sufficient term at an effective dose, for treating EBV infection and EBV-associated disorders, wherein said pharmaceutical agent comprises an antibody or a fragment thereof against HERV-K18 env together with a pharmaceutically acceptable carrier.

### Definitions

The term “EBV-associated disorder(s)”, as used herein, refers to any disease or disorder caused directly or indirectly by EBV, including, but not limited to, X-linked lymphoproliferative syndrome (XLP), nasopharyngeal carcinoma, Burkitt's Lymphoma, Hodgkin's Disease, breast cancer, AIDS-related lymphomas, oral hairy leukoplakia, lupus, rheumatoid arthritis and Sjorgen's syndrome among others.

The term “nucleic acid”, as used herein, refers to either DNA or RNA, including complementary DNA (cDNA), genomic DNA and messenger RNA (mRNA). As used herein, “genomic” means both coding and non-coding regions of the isolated nucleic acid molecule. “Nucleic acid sequence” refers to a single- or double-stranded polymer of deoxyribonucleotide or ribonucleotide bases read from the 5′ to the 3′ end. It includes both self-replicating plasmids, infectious polymers of DNA or RNA, including viral nucleic acids, and nonfunctional DNA or RNA.

The term “polypeptide”, as used herein, refers to either the full length gene product encoded by the nucleic acid, or portions thereof. Thus, “polypeptide” includes not only the full-length protein, but also partial-length fragments, including peptides less than fifty amino acid residues in length.

The phrase “nucleic acid molecule encoding” refers to a nucleic acid molecule which directs the expression of a specific polypeptide. The nucleic acid sequences include both the DNA strand sequence that is transcribed into RNA, the complementary DNA strand, and the RNA sequence that is translated into protein. The nucleic acid molecule includes both the full length nucleic acid sequence as well as non-full length sequences. It being further understood that the sequence includes the degenerate codons of the native sequence or sequences which may be introduced to provide codon preference in a specific host cell.

The term “pharmaceutical composition” refers to preparations which are in such form as to permit the biological activity of the active ingredients to be unequivocally effective, and which contain no additional components which are toxic to the subjects to which the composition would be administered. Such pharmaceutical compositions may be prepared and formulated in dosage forms by methods known in the art; for example, see Remington's Pharmaceutical Sciences, Mack Publishing Company, Easton, Pa., 15th Edition 1975.

“Pharmaceutically acceptable” excipients (vehicles, additives) are those which can reasonably be administered to a subject mammal to provide an effective dose of the active ingredient employed. Typical vehicles include saline, dextrose solution, Ringer's solution, etc. but non-aqueous vehicles may also be used.

In a pharmacological sense, in the context of the present invention, an “effective amount” of the antibody, such as an anti-HERV-K18 env antibody refers to an amount effective in control of EBV-associated condition. In this context, the term “control” is used to include both prophylaxis and treatment of such disorders. Accordingly, the antibody may be administered prophylactically (i.e prior to the appearance of the infection or disorder), or therapeutically (i.e. after appearance of the infection or disorder).

Unless otherwise defined, all technical and scientific terms used herein have the same meaning as commonly understood by one of ordinary skill in the art. Although methods and materials similar or equivalent to those described herein can be used in the practice or testing of the invention, the preferred methods and materials are described below. All publications, patent applications, patents and other references mentioned herein are incorporated by reference. In addition, the materials, methods and examples are illustrative only and not intended to be limiting. In case of conflict, the present specification, including definitions, controls.

### BRIEF DESCRIPTION OF THE DRAWINGS

The accompanying drawings, which are incorporated in and constitute a part of this specification, illustrate embodiments of the invention and, together with the description, serve to explain the objects, advantages, and principles of the invention.

FIGS. 1a-c illustrate that HERV-K18 env alleles preferentially activate hTCRBV13 and hTCRBV9 THys, as does the EBV-associated SAg. FIG. 1a shows the IL-2 production in response to untransfected A20 cells, or five individual clones of A20 stably transfected with HERV-K18.2 env, pretreated with PMA, then resuspended with an equal number of hTCRBV13S1 or hTCPBV8 THy. The IL-2 response was compared to PMA treated B95-8 transformed B LCL from the K18.2 env donor, and to the maximal IL-2 production obtained by anti-CD3 crosslinkage. FIGS. 1b-h show the IL-2 production in response to A20 transfected with HERV-K18 env alleles 1 or 2, or vector only (A20/K18.1 env, A20/K18.2 env, A20/pCDLI, respectively); B95-8 transformed LCL, BL41 and BL41/B95-8 infected cells were pretreated with PMA/mitomycin C, and resuspended with the indicated hTCRBV THy at APC:responder ratios of 5:1 (black bars) or 1:1 (white bars). The results are expressed as percentage maximal IL-2 production based on the stimulation of each THy by anti-CD3 crosslinkage. Maximal IL-2 production (pg/ml) for this assay: TCRBV2=389.1±108.2; TCRBV3=255.7±16.3; TCRBV8=497.2±11.7; TCRBV9=34.1±14.7; TCRBV13S1=141.2±13.5 TCRBV13S2=19.8±9.9; and TCRBV17S1=58.05±36.9.

FIGS. 1i-m illustrate that anti-HERV K18 env antiserum and MHC class II antibodies block activation of THy by K18 env transfectants and the EBV-associated SAgs. FIGS. 1i & 1j shows the IL-2 production in response to PMA/mitomycin-C-pretreated A20/K18.1 env, A20/K18.2 env incubated with Env antiserum, diluted 1:100 or 1:200, or with preimmune serum (1:100) for 30 min prior to addition of hTCRBV 13S 1 THy or hTCRBV13S2 at an APC/responder ratio of 2:1. IL-2 production was measured after 24 hr. The response was compared with A20/pCDLI (negative control). FIGS. 1k & 1l show the IL-2 production in response to PMA/mitomycin-C-pretreated B95-8 marmoset cells, B95-8 LCL, BL41, or BL41/B95-8 incubated with the hTCRBV13S1 THy or hTCRBV13S2 Thy. B95-8 LCL and BL41/B95-8 were also pretreated with Env antiserum, diluted 1:100 or 1:200, or with preimmune serum (1:100) for 30 min prior to addition of hTCRBV13S1 THy or hTCRBV13S2 at an APC/responder ratio of 2:1. IL-9 production was measured 24 hr later. The responses were compared with those elicited by anti-CD3 crosslinkage. As toxicity control, the Env antiserum was also added to anti-CD3 wells. FIG. 1m shows the IL-2 production in response to PMA-pretreated A20, A20/K18.1 env, or B95-8 LCL preincubated with antibodies specific for HLA.DR, H-2D^{d}, I-A^{d}, or I-E^{k/d} and then added to hTCRBV13 S1 at an APC/responder ratio of 1:1. IL-2 production was measured after 24 hr.

FIGS. 2a-c illustrate that B95-8 EBV transcriptionally activates HERV-K18 env expression in B cells. FIG. 2a shows total RNA from B95-8 transformed LCL, BL41, and BL41/B95-8 infected cells, treated for 0, 2, 8, of 16 h with PMA, incubated with riboprobes specific for HERV-K18 env alleles and hTBP (loading control), then digested with RNases and run on a 6% polyacrylamide gel. Protected fragments for HERV-K18 env were detected at 300 b and for hTBP as a doublet at 161 b. The 200 b doublet represents a partial digests of hTBP. As controls, RNA from A20 and A20 transfected with HERV K18.1 were included. The a20/K18.1 env construct has an additional 30 b of Bluescript vector sequence that is protected by the riboprobe, accounting for the difference in size between positive control and the 300 b K18 env band. Densitometry value ratios for the K18 env: hTBP doublet are indicated below each lane. FIG. 2b shows a relative quantitative RT-PCR that was performed using RNA derived from purified B cells from three different donors (1-3) and B95-9 transformed B LCL from the same three donors. Primers were designed to detect a 161 bp HERV K18 read-through transcript that traverses the env gene, 3′ LTR, and adjacent chromosome 1 sequences located up to 122 bp downstream of the 3′ LTR. 25 PCR cycles were determined to yield product within the linear range. Because the read-through transcripts were extremely rare, PCR was performed in the presence of [³²P] α-dCTP. As endogenous standard, primers specific for an 18s rRNA 489 bp product were used in each reaction; and as negative controls, H₂O only and no RT reactions were simultaneously performed. PCR products were separated on a 6% denaturing acrylamide gel and quantified by Phosphorimaging. The ratios of HERV K18:18s rRNA are printed below each lane, and the fold induction of HERV K18 transcripts after B95-8 transformation is depicted for each individual (B95-8:B). FIG. 2c shows the IL-2 production in response to purified primary B cells from three individuals treated with LPS and compared with B95-8-transformed B LCL derived from the same blood donors. Both LPS B cells and B95-S LCL were pretreated with PMA, washed, and incubated with the hTCRBV 13 S1 THy at various APC/responder ratios using 2×10⁴ THy per quadruplicate well. IL-2 production was measured 48 hr later.

FIGS. 3a and 3b illustrate that the EBV-associated SAg activity is caused by K18 env. FIG. 3a shows that A20 transfected with K18.1 env activated peripheral blood T cells with kinetics and magnitude similar to the EBV-associated SAg. PMA/mitomycin C treated A20/K18.1 env or A20/pCDLI, and autologous B95-8 transformed LCL were used as APC in 48 hr T cell proliferation assays, as measured by the incorporation of [³H]thymidine, APC:responder ratios of 1:1 (black bars), 1:3 (grey bars), and 1:10 (white bars), show that T cell proliferation is dependent upon antigen dose. The response is compared to the mitogen PHA, and APC are only shown for comparison. FIG. 3b shows that K18 emv anti-peptide (a.a. 116-130) antiserum blocked 48 h T cell proliferation to PMA/mitomycin C treated A20/K18.1, preincubated at 1:100 and 1:200 dilutions, while preimmune serum did not. In addition, T cell proliferation to autologous B95-8 transformed LCL from an EBV seronegative donor was blocked by the env antiserum, but not the preimmune serum, while the env antiserum had no effect on T cell proliferation due to PHA. The B95-8 marmoset cell line, which produces high titer EBV, was not stimulatory to the EBV seronegative donor T cells.

FIG. 4 shows HERV-K18 env amino acid sequence of SEQ ID:1, SEQ ID:3, and SEQ ID:4.

### DESCRIPTION OF THE INVENTION

We have identified a possible causal agent of EBV-associated disorders in humans as EBV-mediated transactivation of human endogenous retrovirus HERV-K18 env with superantigen (SAg) activity capable of stimulating large fractions of T cells. This transactivation of endogenous SAg may facilitate progression of EBV infection towards a lifelong viral persistence which, under conducive conditions, may result in numerous disorders such as infectious mononucleosis (IM), EBV-induced lymphomas, EBV-associated autoimmune diseases such as lupus, rheumatoid arthritis and Sjogren's syndrome.

### Vaccines and Prophylaxis for EBV Infection and EBV-Associated Disorders

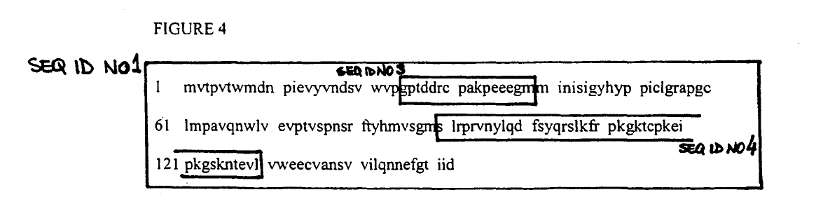

The present invention provides substances suitable for use as vaccines for the prevention of EBV infection and EBV-associated disorders and methods for administering them. The vaccines are directed against HERV-K18 env (SEQ ID:1) and most preferably comprise antigens obtained from HERV-K18 env. Preferred antigens include SEQ ID:2 (cpkeipkgskntevl), SEQ ID:3 and SEQ ID:4 (see FIG. 4). Most preferably, the SAg T cell stimulatory activity of the HERV-K18 env is diminished or eliminated. In another embodiment, the vaccine contains a nucleic acid encoding HERV-K18 env or an immunogenic fragment thereof.

This invention provides a method of vaccinating a subject against EBV and EBV-associated disorders, comprising administering to the subject an effective amount of HERV-K18 env (SEQ ID:1 (see FIG. 4)) or an immunogenic fragment thereof, or a nucleic acid encoding the antigen, and a suitable acceptable carrier, thereby vaccinating the subject. One or more boosts may be administered.

The vaccine can be made using synthetic peptide or recombinantly-produced polypeptide described above as antigen. Typically, a vaccine will include from about 0.1 to 1 mg of antigen. Typically, the vaccine is formulated so that a dose includes about 0.5 milliliters. The vaccine may be administered by any route known in the art. Preferably, the route is parenteral. More preferably, it is subcutaneous or intramuscular.

There are a number of strategies for amplifying an antigen's effectiveness, particularly as related to the art of vaccines. For example, cyclization or circularization of a peptide can increase the peptide's antigenic and immunogenic potency. See U.S. Pat. No. 5,001,049. More conventionally, an antigen can be conjugated to a suitable carrier, usually a protein molecule. This procedure has several facets. It can allow multiple copies of an antigen, such as a peptide, to be conjugated to a single larger carrier molecule. Additionally, the carrier may possess properties which facilitate transport, binding, absorption or transfer of the antigen.

For parenteral administration, such as subcutaneous injection, examples of suitable carriers are the tetanus toxoid, the diphtheria toxoid, serum albumin and lamprey, or keyhole limpet hemocyanin because they provide the resultant conjugate with minimum genetic restriction. Conjugates including these universal carriers can function as T cell clone activators in individuals having very different gene sets.

The conjugation between a peptide and a carrier can be accomplished using one of the methods known in the art. Specifically, the conjugation can use bifunctional cross-linkers as binding agents as detailed, for example, by Means and Feeney, “A recent review of protein modification techniques,” Bioconjugate Chem. 1:2-12 (1990).

The vaccines may be administered by any conventional method for the administration of vaccines including oral and parenteral (e.g., subcutaneous or intramuscular) injection. Intramuscular administration is preferred. The treatment may consist of a single dose of vaccine or a plurality of doses over a period of time. It may be preferred that the dose be given to a human patient within the first 8 months of life.

Those of skill will readily recognize that it is only necessary to expose a mammal to appropriate epitopes in order to elicit effective immunoprotection. The epitopes are typically segments of amino acids which are a small portion of the whole protein. Using recombinant genetics, it is routine to alter a natural protein's primary structure to create derivatives embracing epitopes that are identical to or substantially the same as (immunologically equivalent to) the naturally occurring epitopes. Such derivatives may include peptide fragments, amino acid substitutions, amino acid deletions and amino acid additions.

### Administration

The subjects to be treated may be a mammal, or more specifically a human, horse, pig, rabbit, dog, monkey, or rodent. In the preferred embodiment the subject is a human.

The compositions are administered in a manner compatible with the dosage formulation, and in a therapeutically effective amount. Precise amounts of active ingredient required to be administered depend on the judgment of the practitioner and are peculiar to each subject.

Suitable regimes for initial administration and booster shots are also variable, but are typified by an initial administration followed by repeated doses at one or more hour intervals by a subsequent injection or other administration.

As used herein “administration” means a method of administering to a subject. Such methods are well known to those skilled in the art and include, but are not limited to, administration topically, parenterally, orally, intravenously (i.v.), intramuscularly (i.m.), subcutaneously or by aerosol. Administration of the agent may be effected continuously or intermittently such that the therapeutic agent in the patient is effective to treat a subject with an EBV-associated disorder.

The pharmaceutical formulations or compositions of this invention may be in the dosage form of solid, semi-solid, or liquid such as, e.g., suspensions, aerosols or the like. Preferably the compositions are administered in unit dosage forms suitable for single administration of precise dosage amounts. The compositions may also include, depending on the formulation desired, pharmaceutically-acceptable, nontoxic carriers or diluents, which are defined as vehicles commonly used to formulate pharmaceutical compositions for animal or human administration. The diluent is selected so as not to affect the biological activity of the combination. Examples of such diluents are distilled water, physiological saline, Ringer's solution, dextrose solution, and Hank's solution. In addition, the pharmaceutical composition or formulation may also include other carriers, adjuvants; or nontoxic, nontherapeutic, nonimmunogenic stabilizers and the like. Effective amounts of such diluent or carrier are those amounts which are effective to obtain a pharmaceutically acceptable composition in terms of solubility of components, or biological activity, etc.

### Immunological Therapy

There is provided an article of manufacture comprising packaging material and a pharmaceutical agent contained within said packaging material, wherein said packaging material comprises a label which indicates said pharmaceutical may be administered, for a sufficient term at an effective dose, for treating EBV infection and EBV-associated disorders, wherein said pharmaceutical agent comprises an antibody or a fragment thereof against HERV-K18 env together with a pharmaceutically acceptable carrier.

The antibody may be administered to a patient either singly or in a cocktail containing two or more antibodies, other therapeutic agents, compositions, or the like, including, but not limited to, immunosuppressive agents, potentiators and side-effect relieving agents. All of these agents are administered in generally accepted efficacious dose ranges such as those disclosed in the Physician Desk Reference (2000), Publisher Edward R. Barnhart, New Jersey.

The antibody may be formulated into an injectable preparation. Parenteral formulations are known and are suitable for use in the invention, preferably for i.m. or i.v. administration. The formulations containing therapeutically effective amounts of antibodies are either sterile liquid solutions, liquid suspensions or lyophilized versions and optionally contain stabilizers or excipients. Lyophilized compositions are reconstituted with suitable diluents, e.g., water for injection, saline, 0.3% glycine and the like, at a level of about from 0.01 mg/kg of host body weight to 10 mg/kg where appropriate. Typically, the pharmaceutical compositions containing the antibodies will be administered in a therapeutically effective dose in a range of from about 0.01 mg/kg to about 5 mg/kg of the treated individual. A preferred therapeutically effective dose of the pharmaceutical composition containing antibody will be in a range of from about 0.01 mg/kg to about 0.5 mg/kg body weight of the treated individual administered over several days to two weeks by daily intravenous infusion, each given over a one hour period, in a sequential patient dose-escalation regimen.

Antibody may be administered systemically by injection i.m., subcutaneously or intraperitoneally. The dose will be dependent upon the properties of the antibody employed, e.g., its activity and biological half-life, the concentration of antibody in the formulation, the site and rate of dosage, the clinical tolerance of the patient involved, the disease afflicting the patient and the like as is well within the skill of the physician.

The antibody of the present invention may be administered in solution. The pH of the solution should be in the range of pH 5 to 9.5, preferably pH 6.5 to 7.5. The antibody or derivatives thereof should be in a solution having a suitable pharmaceutically acceptable buffer such as phosphate, tris (hydroxymethyl) aminomethane-HCl or citrate and the like. Buffer concentrations should be in the range of 1 to 100 mM. The solution of antibody may also contain a salt, such as sodium chloride or potassium chloride in a concentration of 50 to 150 mM. An effective amount of a stabilizing agent such as an albumin, a globulin, a gelatin, a protamine or a salt of protamine may also be included and may be added to a solution containing antibody or immunotoxin or to the composition from which the solution is prepared.

Systemic administration of antibody is made daily, generally by intramuscular injection, although intravascular infusion is acceptable. Administration may also be intranasal or by other nonparenteral routes. Antibody may also be administered via microspheres, liposomes or other microparticulate delivery systems placed in certain tissues including blood.

The antibodies may be raised against either a peptide of or the whole molecule. Such a peptide may be presented together with a carrier protein, such as an KLH, to an animal system or, if it is long enough, say 25 amino acid residues, without a carrier.

Polyclonal antibodies generated by the above technique may be used direct, or suitable antibody producing cells may be isolated from the animal and used to form a hybridoma by known means (Kohler and Milstein, Nature 256:795. (1975)). Selection of an appropriate hybridoma will also be apparent to those skilled in the art.

It will be appreciated that antibodies for use in accordance with the present invention may be monoclonal or polyclonal as appropriate. Antibody equivalents of these may comprise: the Fab′ fragments of the antibodies, such as Fab, Fab′, F(ab′)2 and Fv; idiotopes; or the results of allotope grafting (where the recognition region of an animal antibody is grafted into the appropriate region of a human antibody to avoid an immune response in the patient), for example. Single chain antibodies may also be used. Other suitable modifications and/or agents will be apparent to those skilled in the art.

Chimeric and humanized antibodies are also within the scope of the invention. It is expected that chimeric and humanized antibodies would be less immunogenic in a human subject than the corresponding non-chimeric antibody. A variety of approaches for making chimeric antibodies, comprising for example a non-human variable region and a human constant region, have been described. See, for example, Morrison et al., Proc. Natl. Acad. Sci. U.S.A. 81,6851 (1985); Takeda et al., Nature 314,452(1985), Cabilly et al., U.S. Pat. No. 4,816,567; Boss et al., U.S. Pat. No. 4,816,397; Tanaguchi et al., European Patent Publication EP 171496; European Patent Publication 0173494, United Kingdom Patent GB 2177096B. Additionally, a chimeric antibody can be further “humanized” such that parts of the variable regions, especially the conserved framework regions of the antigen-binding domain, are of human origin and only the hypervariable regions are of non-human origin. Such altered immunoglobulin molecules may be made by any of several techniques known in the art, (e.g., Teng et al., Proc. Natl. Acad Sci. U.S.A., 80, 7308-7312 (1983); Kozbor et al., Immunology Today, 4,7279 (1983); Olsson et al., Meth. Enzymol., 92, 3-16 (1982)), and are preferably made according to the teachings of PCT Publication WO92/06193 or EP 0239400. Humanized antibodies can be commercially produced by, for example, Scotgen Limited, 2 Holly Road, Twickenham, Middlesex, Great Britain. Antibodies for use in accordance with the present invention may also be prepared using the methods described in U.S. Pat. No. 6,111,166, incorporated herein by reference in its entirety.

Another method of generating specific antibodies, or antibody fragments, reactive against EBV is to screen phage expression libraries encoding immunoglobulin genes, or portions thereof, with a protein of the invention, or peptide fragment thereof. For example, complete Fab fragments, V H regions and V-region derivatives can be expressed in bacteria using phage expression libraries. See for example Ward, et al., Nature 341,544-546: (1989); Huse, et al., Science 246, 1275-1281 (1989); and McCafferty, et al., Nature 348, 552-554 (1990).

The invention will be further characterized by the following examples which are intended to be exemplary of the invention.

### EXAMPLES

### Experimental Techniques

We found that EBV transactivates the human endogenous retrovirus HERV-K18 (8) (which was recently localized to chromosome 1q21.2-q22 in the first intron of CD48) that has SAg activity. An EBV inducible enhancer had been previously mapped to a region 1.58 kb upstream of the CD48 start site. It was also shown that the IDDMK_{1,2}22 retrovirus is an allelic variant of HERV-K18 (designated allele 1 or K18.1), whose env gene encodes SAg activity. These findings led us to test whether any of the HERV-K18 env alleles possessed TCRBV13 SAg activity that could be induced by EBV. Tonjes, R. R. Czauderna, F. & Kurth, R. Genome-wide screening, cloning, chromosomal assignment, and expression of full-length human endogenous retrovirus type K. J Virol 73, 9187-9195 (1999); Thorley-Lawson, D. A., Schooley, R. T., Bhan, A. K. & Nadler, L. M. Epstein-Barr virus superinduces a new human B cell differentiation antigen (B-LAST 1) expressed on transformed lymphoblasts. Cell 30, 415-425 (1982); Klaman, L. D. & Thorley-Lawson, D. A. Characterization of the CD48 gene demonstrates a positive element that is specific to Epstein-Barr virus immortalized B-cell lines and contains an essential NF-kappa B site. J Virol 69, 871-881 (1995); Conrad, B. et al. A human endogenous retroviral superantigen as candidate autoimmune gene in type I diabetes. Cell 90, 303-313 (1997); Barbulescu, M. et al. Many human endogenous retrovirus K (HERV-K) proviruses are unique to humans. Curr Biol 9, 861-868 (1999).

The HERV-K18 env alleles 1 and 2, K18.1 and K18.2, differ at several positions; the K-18.1 Env has a stop codon at a.a. 153, while the K18.2 Env is a full length 553 amino acid protein. Since the K18.1 allele had been previously characterized, we tested whether the full length env of K18.2 could stimulate T cells. We therefore cloned the entire HERV-K18.2 provirus, using as PCR primers the chromosome 1 insertion sequences previously reported. After sequencing, the env, gene was subcloned into the bicistronic expression vector pCDLI with the marker EYFP (enhanced yellow fluorescent protein) in the second cistron. Murine A20 B lymphoma cells were chosen for transfection experiments, because the mouse genome does not have any HERV related proviruses. Fleischer, B., Necker, A., Leget, C., Malissen, B. & Romagne, F. Reactivity of mouse T-cell hybridomas expressing human Vbeta gene segments with staphylococcal and streptococcal superantigens. Infect Immun 64, 987-994 (1996). Stable clones expressing different levels of EYFP were selected by flow cytometry and tested for TCRBV 13 T cell activation.

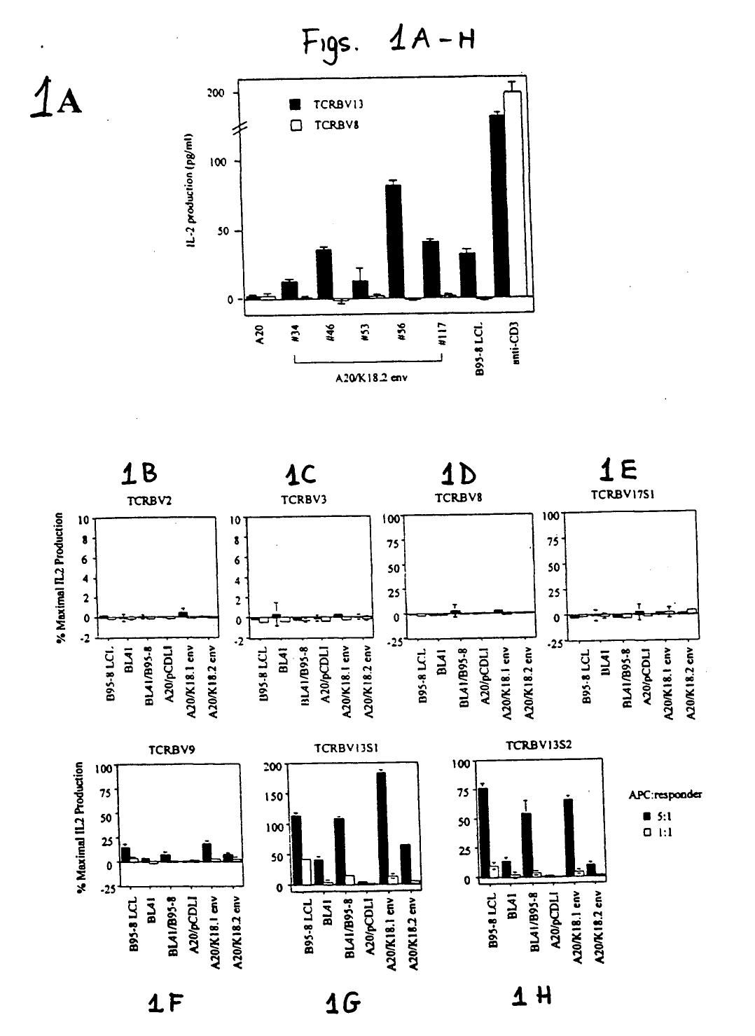

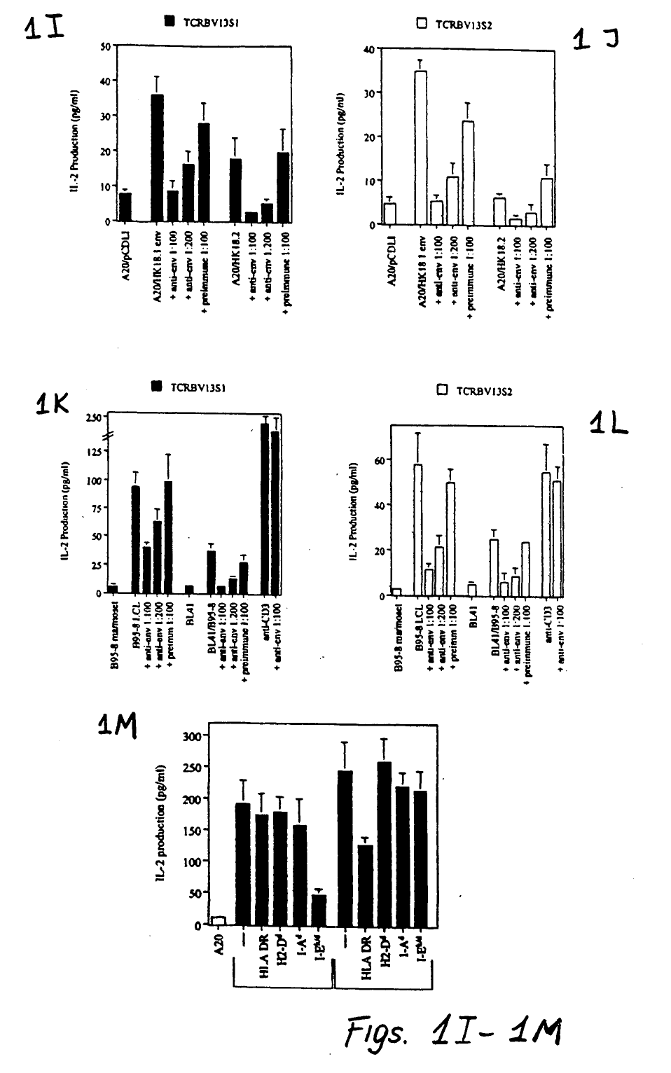

We have previously described a system to assay for EBV-associated SAg activity based on the stimulation of murine T cell hybridomas (THys) with EBV-infected B cell lines acting as antigen presenting cells (APC). These THys bear chimeric TCR composed of a human (h) TCRBV gene product with murine a chain and CD3 proteins (note 1). As can be seen in FIG. 1a, all of the K18.2 env transfectants (A20/K18.2) stimulated the hTCRBV 13 THy, but not the hTCRBV8 THy, whereas both THys were equally activated by CD3 crosslinking. The magnitude of the response was similar to that elicited by a lymphoblastoid cell line (LCL) made from B cells from a K18.2 donor transformed by the B95-8 strain of EBV, while untransfected A20 cells gave no response. Similar results were obtained by transfecting K18.2 env into the human EBV⁻ B cell lymphoma BJAB (data not shown). These data indicate that the K18.2 env allele is recognized by TCRBV 13 similar to the EBV-associated SAg.

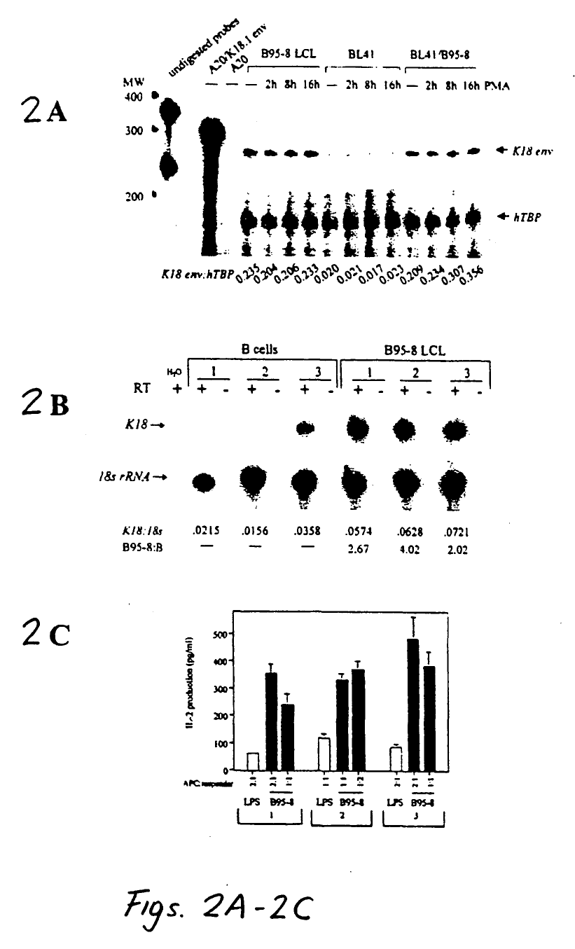

To test whether the K18.2 env transfectants stimulated other T cell subsets, we used a panel of murine THys expressing different hTCRBV genes. In addition, we examined the response to the truncated K18.1 env transfected into A20 cells. The results (note 2), depicted in FIGS. 1b-h, show the comparison between the response obtained with a B95-8 LCL and B95-8 infected Burkitt's lymphoma (BL) BL41 versus uninfected BL41 cells. The EBV⁺ BL and LCL and both K18 env alleles expressed in A20 stimulated the hTCRBV13S1 and hTCRBV13S2 THys, but not the hTCRBV2, 3, 8, or 17 THys, while A20 transfected with pCDLI vector alone did not stimulate any of the hybridomas. At APC:responder ratios of 5:1, the K18 Env alleles and B95-8 infected BL41 and LCL also stimulated the hTCRBV9 THy, suggesting an additional specificity. In addition, uninfected BL41 at high APC ratios weakly stimulated the very sensitive hTCRBV13 S1 THy, most likely due to the low level of endogenous K18 env expression in these cells (see FIGS. 2a-c). It should be mentioned that pretreatment of all APC lines with the phorbol ester PMA was necessary for stimulation of the THys, as was previously shown for the EBV-associated SAg activity. These data show that both K18 env alleles have the same TCRBV specificity as the EBV-associated SAg.

To test whether the SAg activity was due to K18 Emv, we employed a rabbit antiserum raised against the K18 env peptide 116-130, selected by the hydrophilicity index of Kyte and Doolittle. This antiserum specifically blocked immune recognition of the K18.1 and K18.2 env alleles by the TCRBV13S1 and TCRBV 13S2 THys in a dose dependent manner (FIGS. 1i&j), while the preimmune serum had no effect (note 3). We then used this antiserum to prove that the TCRBV13 activation by EBV infected cells was mediated by K18 env. As shown in FIGS. 1k&l, the env antiserum blocked stimulation of these THys by EBV transformed LCL and EBV infected BL41.

On the other hand, the Env antiserum had no effect on the anti-CD3 response, and nonspecific blocking was not observed with the preimmune serum. Moreover, the marmoset cell line B95-8, which expresses both EBV latent and lytic genes and produces high titers of virus, but does not contain the HERV-K18 provirus, did not stimulate the TCRBV13 THys. These data provide evidence that the TCRBV13 specific EBV SAg activity is due to the env gene product of the endogenous HERV-K18 provirus.

To test whether EBV could upregulate HERV-K18 env expression, as it does CD48, we used a RNase protection assay (note 4), designed to detect all of the K18 env alleles, but not other HERV-K env transcripts. As can be seen in FIGS. 2a-c, the emv transcripts are readily detected in a B95-8 EBV transformed LCL and are also highly upregulated when EBV BL41 cells are converted to EBV⁺ by infection with B95-8 virus. Treatment of the APC with PMA had no effect on K18 env transcription. Thus, the PMA enhancement of SAg activity does not work at the level of K18 env transcription. More likely, PMA is acting to increase the efficacy of SAg presentation, perhaps through upregulation of MHC class II or accessory molecules. These data show that EBV transcriptionally activates K18 env expression.

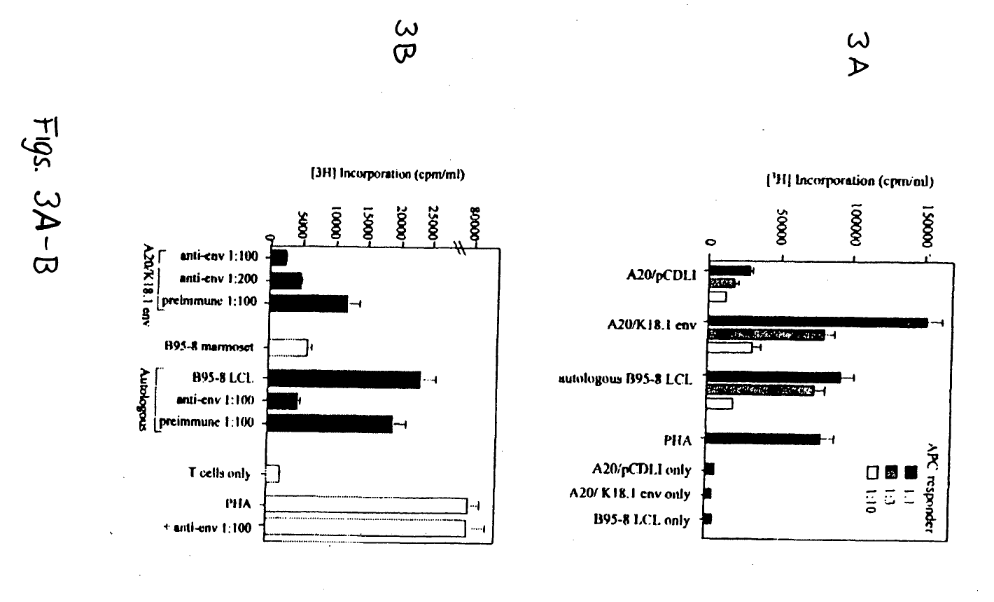

To confirm the stimulatory activity of K18 env on primary T cells, we measured proliferation of peripheral blood T cells induced by A20 cells that were transfected with K18.1 env. Proliferation was assessed 48 h after co-culture (note 5). As can be seen in FIG. 3a, PMA/mitomycin C pretreated A20/K18.1 env vigorously and rapidly stimulated T cells, while pretreated A20/pCDLI conferred only minimal activity. The response was comparable to that elicited with autologous B95-8 transformed LCL, as was previously shown for EBV-associated SAg activity, or the mitogen PHA. To demonstrate that EBV induction of K18 env was driving this polyclonal proliferation, we again performed antibody blocking experiments (note 6) using the rabbit antiserum raised against the K18 env peptide (FIG. 3b). The antiserum blocked peripheral blood T cells from responding to A20/K18.1 env in a dose dependent manner, while preimmune serum was not inhibitory. The env antiserum also completely blocked the T cell proliferative response of an EBV seronegative donor to autologous LCL derived from in vitro transformation of B cells with B95-8 EBV, while the response to the mitogen PHA was unaffected. In addition, these data exclude the possibility that the elicited T cell proliferation was due to a potent recall response. Moreover, no response by this EBV donor was seen to the EBV⁺ marmoset cell line B95-8, similar to the results obtained with the THys (FIGS. 1k&l). It is interesting that marmosets, although easily infected with EBV, do not establish persistent infection. It is thus possible, that the SAg activity elicited by HERV-K18 env upon EBV infection is required for the long-term latency of EBV in the host.

We have shown that EBV infection of B cells leads to transactivation of HERV-K18 env alleles, which express a TCRBV 13 specific SAg activity, previously identified as an EBV-associated SAg. Sutkowski, N. et al. An Epstein-Barr virus-associated superantigen. J Exp Med 184, 971-980 (1996). This represents the first demonstration of a microbial pathogen inducing an endogenous SAg for its own use. It will be interesting to study the interplay of biological activity that has allowed the evolutionary retention of an endogenous retrovirus that potentially benefits a persistent herpesvirus. Detection of this SAg activity required a highly defined system whereby murine transfectants presented the K18 env gene product to hTCRBV specific THys. The chimeric human/mouse TCR of the THys revealed the preference for TCRBV 13.1, 13.2 and 9 gene products. In primary cells the EBV-associated T cell response, while initially TCRBV13 restricted, rapidly became polyclonal. Indeed, we have shown here that K18 env induced a polyclonal response in peripheral blood T cells, whether presented by mouse APC or EBV infected B cells. Similar effects have been seen in toxin titration experiments with bacterial SAgs and might account for controversy over the initial finding of TCRBV7 specificity of K18.1 Env.

Thus, in vivo, EBV infection leads to expression of an endogenous provirus with powerful T cell stimulators activity has widespread implications for understanding EBV pathogenesis. Extensive T cell infiltrates are characteristic of the EBV-associated tumors Hodgkin's lymphoma and naso-pharyngeal carcinoma; and there is good evidence for a role of activated T helper cells in the development of transplant associated lymphomas. Furthermore, massive lymphocytosis is characteristic of acute infectious mononucleosis. EBV induced SAg activity could play a role in any of these processes.

The following notes and references are cited throughout the specification and are incorporated herein by reference.

Notes

(Note 1) All cell lines were grown in RPMI (Gibco) supplemented with 10% FCS, glutamine, HEPES, Na pyruvate, β-mercaptoethanol. EBV cell lines and stable A20 transfectants expressing HERV-K18.2 ells, were treated overnight with PMA (Calbiochem, 10 ng/ml) at 37° C., then with mitomycin C (Sigma, 0.1 mg/ml) for 1 h, and washed extensively with PBS. Cells were counted and resuspended with THy in quadruplicate wells of 96 well round bottom plates, using 2×10⁴ of each cell type/well. After 48 h at 37° C., the plates were frozen at −80° C. to lyse the cells, and thawed supernatants were tested for the presence of mIL-2 by ELISA (Pharmingen), and compared to a standard curve with rIL-2 (R&D Systems). As positive control, the THy were stimulated with platebound anti-CD3 (145 2C 11, Pharmingen).

(Note 2) A20 transfected with K18.1 or K18.2 env, or pCDLI, and EBV cell lines were PMA/mitomycin C treated as above, and resuspended at APC:responder of 5:1 or 1:1 with THy, using 2×10⁴ THy/well. IL-2 production for each THy was expressed as % maximal based on the response to platebound anti-CD3.

(Note 3) Antiserum blocking studies were performed by preincubating APC for 30 min at 37° C. with rabbit anti-Env peptide 116-130 antiserum diluted 1:100 or 1:200, or preimmune serum at 1:100. APC:responder ratio was 2:1, with 2×10⁴ THy per well. Plates were frozen at 24 h, and thawed supernatants were tested for mIL-2 as above.

(Note 4) 2×10⁸ BL41, BL41/B95-8 (a from G. Lenoir) or B95-8 LCL (made by transforming 10⁶ peripheral blood B cells with 1 ml of 5 d B95-8 virus supernatant, diluted 1:1 in media, for 1.5 h at 37° C., then expanded for several weeks in 10% FCS/complete RPMI media), were treated for 0, 2, 8 or 16 h with PMA (10 ng/ml), then total RNA was prepared with Trizol (Gibco BRL). The RNase protection assay was performed as previously described, but with 100 μg total RNA/lane. As controls, 100 μg RNA from untransfected A20 cells, and 20 μg RNA from A20 transfected with HERV-K18.1 env (IDDM465) were loaded on the gel. (It should be noted that this transfectant vastly overexpressed the env gene compared to LCL). Densitometry values were obtained by scanning the autoradiograph with a Biorad Gel Doc 1000, using Molecular Analyst program. The ratio of K18 env: hTBP (human TATA binding protein) was determined.

(Note 5) Peripheral blood mononuclear cells were obtained from healthy adult volunteers, plated overnight at 37° C. in 10% FCS/complete RPMI media to allow monocytes to adhere and then used as a source of T cells. A20 transfected with HERV-K18.1 env or pCDLI only or B95-8 LCL, transformed from autologous B cells, were treated overnight with PMA (10 ng/ml), then with mitomycin C (0.1 mg/ml) for 1 h, and washed extensively with PBS. APC and T cells were resuspended at various ratios, using 10⁵ T cells per well in quadruplicate in 96 well round bottom plates. After 48 h at 37° C. cells were pulsed with (3H)thymidine (1 μCi/well) for 12 h, then harvested and counted for (³H) incorporation.

(Note 6) Antiserum blocking studies were performed identically; however, prior to addition of T cells, APC were preincubated for 30 min with Env antiserum diluted 1:100 or 1:200, or preimmune serum at 1:100.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

The references appearing throughout the application are incorporated herein by reference.

We claim:

1. A vaccine for treating and/or preventing EBV infection and EBV-associated disorders comprising HERV-K18 env SEQ ID:1 or an immunogenic fragment thereof, or a nucleic acid encoding the HERV-K18 env, or a fragment thereof and a pharmaceutically acceptable carrier.

2. The vaccine of claim 1, wherein the immunogenic fragment is selected from the group consisting of SEQ ID:2, SEQ ID:3 and SEQ ID:4.

3. The vaccine of claim 1, wherein the immunogenic fragment is SEQ ID:2.

4. An isolated peptide having the amino acid sequence of SEQ ID:2.

5. The vaccine of claim 1, wherein the immunogenic fragment comprises the whole HERV-K18 env protein, or a peptide thereof, in which the superantigen T cell stimulatory activity of HERV-K18 env is diminished.

6. A method for preventing EBV infection and EBV-associated disorders in an individual at risk for said infection comprising administering to said individual the vaccine of claims 1, 2 or 3.

7. A method for treating an individual having an EBV-associated disorder comprising administering to said individual a treatment effective amount of an antibody or a fragment thereof against HERV-K18 env.

8. The method of claim 7, wherein the EBV-associated disorder is infectious mononucleosis or an EBV induced lymphoma.

9. A method for providing passive immunity to infection by EBV in an individual susceptible to infection by. EBV, said method comprising administering to said individual a HERV-K18 env antibody composition.

10. A method for preventing EBV-associated disorders in immunosuppressed individuals comprising administering to said individuals the vaccine of claims 1, 2, and 3.

11. The method of claim 10, wherein the vaccine is administered before commencement of immunosuppressive therapy.

12. The method of claim 7, comprising administering to said individual a treatment effective amount of an antibody or a fragment thereof against HERV-K18 env.

13. The method of claim 7, comprising administering to said individual a treatment effective amount of an antibody or a fragment thereof against HERV-K18 env.

14. A method for treating an EBV-associated autoimmune disorder, the method comprising:
a) identifying an EBV-positive immunocompromised individual; and
b) administering to the immunocompromised individual, an effective amount of an antibody or a fragment thereof against HERV-K18 env.

15. A method for treating oncogenic transformation in an immunocompromised individual, the method comprising:
a) identifying an immunocompromised individual exhibiting clinical symptoms associated with early stage oncogenic transformation; and
b) administering to the immunocompromised individual, an effective amount of an antibody or a fragment thereof against HERV-K18 env.

16. A method of claim 15, wherein the oncogenic transformation results in Hodgkin's lymphoma, Post-transplant-lymphoproliferative disorders, Lympho-proliferative Disorders, EBV-positive lymphomas, EBV-positive breast cancer, Burkitt's lymphoma, and Naso-Pharyngeal-Carcinoma.

17. An article of manufacture comprising packaging material and a pharmaceutical agent contained within said packaging material, wherein said packaging material comprises a label which indicates said pharmaceutical may be administered, for a sufficient term at an effective dose, for treating EBV infection and EBV-associated disorders, wherein said pharmaceutical agent comprises an antibody or a fragment thereof against HERV-K18 env together with a pharmaceutically acceptable carrier.
